(19) United States
(12) Patent Application Publication
(10) Pub. No.: US 2002/0143271 A1
(43) Pub. Date: Oct. 3, 2002

(54) Walled adaptor for use with point-of-care testing kit

(75) Inventors: Jamieson William MaClean Crawford, New York, NY (US); Frank Francavilla, Newton, NJ (US)

Correspondence Address: CASELLA & HESPOS LLP, 274 MADISON AVENUE, SUITE 1703, NEW YORK, NY, 10016-0701, US

(73) Assignee: Becton, Dickinson and Company, Franklin Lakes, NJ (US)

(21) Appl. No.: 10/106,148

(22) Filed: Mar. 25, 2002

Related U.S. Application Data
(60) Provisional application No. 60/280,405, filed on Mar. 30, 2001.
(60) Provisional application No. 60/280,438, filed on Mar. 30, 2001.

(51) Int. Cl. A61B 5/00

(52) U.S. Cl. 600/573

(57) ABSTRACT

An adaptor is provided to facilitate delivery of a fluid specimen from a syringe to a point-of-care testing cartridge. The adaptor includes a tube with an outlet end, an inlet end and a passage extending between the ends. The outlet end of the tube is dimensioned for mating with the entry port of the testing cartridge. The inlet end of the tube is dimensioned for mating with the syringe. The adaptor further includes a support wall extending transversely from the tube and at least one guide wall extending from the support wall. The guide wall is spaced transversely from the outlet of the tube by a distance substantially equal to the distance between a side wall of the testing cartridge and the entry port of the testing cartridge. Thus, the guide wall guides the outlet of the tube into the entry port of the testing cartridge.

### RELATED APPLICATIONS

This application claims priority on U.S. Provisional Patent Appl. No. 60/280,405 and U.S. Provisional Patent Appl. No. 60/280,438 both of which were filed on Mar. 30, 2001.

### BACKGROUND OF THE INVENTION

1. Field of the Invention

The subject invention relates to an adaptor to facilitate the transfer of a specimen from a syringe to a point-of-care testing cartridge.

2. Description of the Related Art

Many medical procedures require diagnostic tests to be performed on a sample of a patient's fluid. Fluid often is collected from a patient by employing a needle holder assembly and one or more evacuated tubes. Fluid also can be collected in a syringe. A syringe may be used with a metallic needle to obtain a fluid sample from a patient. However, syringes often are connected directly to an established arterial or venous line to obtain a fluid sample. The fluid collected in the syringe then may be transferred to a tube. The tubes are labeled carefully and shipped to a laboratory for analysis. The results of the laboratory analysis then are reported back to the health care provider. The results, of course, could be rushed in emergency situations, but absent an emergency would require more then one day between the time the sample is drawn from the patient to the time that the laboratory analysis is reported to the health care provider.

Devices have been developed for performing at least certain diagnostic tests on a sample of fluid at the point-of-care. The point-of-care diagnostic equipment includes a syringe for receiving a sample of fluid from a patient, a small disposable testing cartridge for receiving a portion of the fluid from the syringe and a portable clinical analyzer for analyzing the fluid and outputting the results. Combinations of testing cartridges and portable clinical analyzers are marketed in the United States by i-STAT Corporation, AVL Scientific Corporation and Diametrics Medical, Inc. The systems produced by these and other companies share certain common features. In particular, the testing cartridge of each system typically has a small rectangular housing about 1″×2″ and about 0.25″ thick. The housing includes an internal reservoir with a volume of between about 40 μl and 125 μl. An inlet port extends through an external wall of the testing cartridge and communicates with the internal reservoir. The cartridge further includes contact pads and sensors that can be placed in communication with the portable clinical analyzer. An example of an i-STAT point-of-care testing cartridge is shown in U.S. Pat. No. 5,638,828.

The prior art point-of-care testing systems are employed with a syringe that is used to draw a sample of fluid from a patient. The syringe then may be used to eject a portion of the fluid sample into the inlet port of the point-of-care testing cartridge. However, some testing cartridges are operative to automatically draw fluid from the syringe. The inlet port of the cartridge then is closed and the cartridge is placed in communication with the portable clinical analyzer for performing certain specified diagnostic tests on the sample of fluid in the cartridge. The analyzer then provides a very quick output of the test results without the need for sending the fluid sample to the laboratory.

Point-of-care testing systems provide several efficiencies over systems that require virtually all diagnostic tests to be performed at a location remote from the point-of-care. The small size of the testing cartridge facilitates storage and shipment of the cartridges while also contributing to the portability of the system. However, with regards to transferring a collected sample to the cartridge, the small cartridges can be very difficult to use. For example, alignment of the distal end of the syringe with the inlet port of the testing cartridge can be complicated and difficult. A misalignment or imprecise mating of the syringe with the inlet port of the testing cartridge can lead to a loss of a portion of the collected fluid sample. Additionally, it is difficult to use a syringe for accurately dispensing the proper volume of liquid. Too small a volume may prevent proper testing by the cartridge and the associated portable clinical analyzer. Too large a volume can cause splattering or spillage. Similarly an overfill can result in splatter when the cover of the point-of-care testing cartridge is closed. Fluid that is not delivered efficiently from the syringe into the inlet port of the testing cartridge create the potential for disease transmission. Similarly, a loss of fluid during the transfer from the syringe to the testing cartridge can leave an insufficient volume of fluid for performing the required diagnostic tests. An insufficient volume of fluid to perform the required tests can require the health care worker to return to the patient for a second sample of fluid. This is time consuming for the health care worker and traumatic for the patient. Additionally, some testing cartridges may require an insufficiently filled cartridge to be discarded and a new cartridge to be employed with the new sample of fluid. Thus, inefficiencies in the transfer of fluid from the syringe to the testing cartridge can generate excess costs for additional testing cartridges.

The direct transfer of fluid from a syringe to a testing cartridge can cause the syringe tip to close off the entry port and prevent venting of air from the testing cartridge. Thus bubbles are created. Bubbles reduce the volume of fluid and can affect test results.

IV access systems of tubes and fittings often are used for delivering liquid solutions to a patient. One such fitting is a blunt plastic tube with opposed proximal and distal ends and a lumen extending therebetween. Portions of the lumen adjacent the proximal end of the plastic fitting define a large tapered opening dimensioned to achieve a fluid-tight engagement with the tapered tip of a Luer fitting, such as the tip at the distal end of a syringe. The proximal end of the plastic fitting includes a pair of diametrically opposite lugs that are configured for engagement with the internal threads on a Luer collar. Threaded engagement of the lugs on the plastic fitting with the internal threads of the Luer collar cause the tip of the Luer fitting to telescope tightly into the tapered entry to the lumen of the plastic fitting. Thus, the prior art plastic fitting can achieve a secure mechanical connection with a Luer collar and a fluid-tight connection with the distal tip of the Luer fitting. The extreme distal tip of the plastic fitting terminates in a single axially aligned egress port with a diameter similar to the diameter of the lumen. Thus, the distal end of the plastic fitting is not beveled to a sharp point. Plastic fittings of this type include those sold by Baxter and Becton Dickinson under the trademark INTERLINK®.

Plastic fittings have been used for a variety of medical purposes, including the injection of drugs into the fitting of an IV line. The plastic fittings, however, typically have not been used for phlebotomy or during any diagnostic procedures conducted after a sample of blood has been collected.

### SUMMARY OF THE INVENTION

The subject invention is directed to a walled adaptor for use with a point-of-care testing cartridge and with a syringe assembly. The point-of-care testing cartridge may be a prior art testing cartridge as described above, or any yet-to-be developed testing cartridge for performing point-of-care diagnostic analysis on a collected specimen of blood or other bodily fluid. The testing cartridge comprises a housing having an internal reservoir for receiving a specimen to be tested. The housing may be substantially rectangular, with opposed top and bottom walls and a plurality of side walls. An entry port extends through the top wall and communicates with the internal reservoir of the testing cartridge. The testing cartridge may further include contact pads and sensors that can be placed in communication with a portable clinical analyzer for performing point-of-care analysis of the collected specimen.

The syringe assembly that is used with the walled adaptor may be a conventional prior art syringe assembly. The syringe assembly includes a body with opposed proximal and distal ends. A barrel extends distally from the proximal end of the body and defines a fluid receiving chamber that is widely open at the proximal end. A Luer tip projects from the barrel to the distal end of the syringe body and includes a passage that communicates with the fluid receiving chamber. The Luer tip includes a conically tapered outer surface that is dimensioned and configured for mating with the tapered proximal entry to the hub of a needle assembly or with the base of a plastic blunt Luer fitting or a plastic cannula. The distal end of the syringe body may further have an internally threaded Luer collar that projects from the distal end of the barrel and concentrically around the Luer tip. The threads of the Luer collar can be engaged threadedly with lugs at the proximal end of the hub of a needle assembly or with comparable lugs at the proximal end of a plastic Luer fitting or blunt plastic cannula. Luer tips, Luer collars and mating structures on needles or cannulas are known in the art.

The syringe assembly further includes a plunger that is slidably received in the open proximal end of the fluid receiving chamber defined by the syringe barrel. Distal movement of the plunger in the fluid receiving chamber will expel a fluid from the chamber and through the Luer tip. Proximal movement of the plunger in the chamber will draw fluid through the Luer tip and into the chamber.

The syringe assembly with which the walled adaptor is used may further include a needle assembly a plastic Luer fitting or a blunt plastic cannula for accessing blood or other bodily fluid to be tested. A conventional prior art needle assembly includes an elongate metallic needle cannula having a proximal end, a pointed distal end and a lumen extending between the ends. The prior art needle assembly further includes the plastic hub having opposed proximal and distal ends. The distal end of the hub is securely mounted to the proximal end of the needle cannula. The proximal end of the hub is configured for fluid-tight engagement with the Luer tip. Additionally, the proximal end of the hub may include lugs for threaded engagement with the internal threads on a Luer collar that may be present on the syringe. A plastic Luer fitting or a blunt plastic cannula typically is unitarily molded from a plastic material and has opposite proximal and distal ends and a lumen extending between the ends. The proximal end of the plastic Luer fitting or blunt plastic cannula may have the same shape as the proximal end of the hub for the above-described needle assembly. The distal end of the blunt plastic cannula may be tapered sufficiently to pierce a septum across a fitting on an IV access system or blood collection set.

The walled adaptor of the subject invention may be unitarily molded from a plastic material and comprises a tapered tube with a cross-sectionally small outlet section for mating with the inlet port of the prior art testing cartridge, a cross-sectionally large inlet section for mating with the Luer tip of the syringe and a passage or lumen extending between the inlet and outlet sections. The adaptor further comprises a support that extends substantially transverse to the tapered tube of the adapter. The support wall may be contoured to nest with portions of the top wall of the testing cartridge surrounding the entry port to the testing cartridge. Additionally, the support wall is spaced from the outlet end of the tapered tube by a distance sufficient to prevent over-insertion of the outlet section of the adaptor into the entry port of the testing cartridge. Thus, a uniform flow of the specimen through the adaptor and into the reservoir of the testing cartridge can be assured and damage to the interior of the testing cartridge can be avoided.

The adaptor further comprises at least one guide wall that extends from the support wall. The guide wall is spaced from the outlet port of the adaptor by a sufficient distance to enable the guide wall of the adaptor to nest with a side wall of the testing cartridge. Most testing cartridges have the inlet port in proximity to a comer of the testing cartridge. Thus, the adaptor preferably comprises at least to intersecting guide walls for nesting against intersecting side walls of the testing cartridge in proximity to the inlet port. The guide wall extends from the support wall by a distance that exceeds the extension of the outlet section of the tapered tube from the support wall.

The adaptor may further comprise a tip cap hingedly connected to one of the guide walls. The tip cap can be rotated into an open position where the tip cap is spaced from the outlet section of the tube of the adaptor. The tip cap also can be rotated into a closed position where the tip cap sealingly engages the outlet end of the tube of the adaptor. The surface of the tip cap that engages the outlet section of the tube of the adaptor may include a recess configured to telescope tightly over the outlet section of the tube. The dimensions of the recess may be configured to achieve frictional engagement between the tip cap and the outlet section of the adaptor tube. However, the frictional engagement still permits digital pressure on the tip cap to disengage the tip cap from the outlet section of the adaptor tube for rotating the tip cap into the open position. Preferably, the tip cap is unitarily formed with other sections of the adaptor, and the hinged connection defines a living hinge.

The adaptor can be used by first drawing a specimen of blood or other bodily fluid with a syringe assembly substantially in a conventional manner. For example, the Luer tip of the syringe body may be connected to a fitting of an arterial line or venous line that already is in communication with the patient. Alternatively, a plastic Luer fitting or a blunt plastic cannula mounted to the Luer tip may be placed in communication with the fitting of an IV access system or blood collection set. Still further, a conventional needle assembly may be mounted to the Luer tip of the syringe body and the distal tip of the needle cannula can be inserted into a blood vessel of the patient to obtain the required specimen. With any of these approaches, blood is drawn through the passage of the Luer tip and into the fluid receiving chamber of the syringe body by pulling the plunger of the syringe assembly in a proximal direction. Most point-of-care testing cartridges require between 40 μl and 125 μl to complete a test. Hence, the plunger of the syringe assembly is moved proximally to obtain a volume of blood in excess of the amount required by the particular testing cartridge that will be employed.

After the appropriate volume of blood has been collected, the needle assembly, if used, is removed in an accepted safe manner and deposited in a sharps receptacle. Alternatively, any blunt plastic cannula that may have been mounted to the distal end of the syringe body is removed and discarded into a sharps receptacle in a conventional accepted safe manner.

The adaptor of the subject invention then is mounted to the distal end of the syringe body. More particularly, the Luer tip of the syringe body may be urged into fluid-tight frictional engagement with the Luer-tapered inlet to the adapter. Alternatively, the syringe body may include a Luer collar with an array of internal threads and the inlet of the adaptor may include a mating pair of Luer projections. In this situation, the adaptor is threaded into engagement with the Luer collar while simultaneously urging the Luer tip of the syringe into fluid-tight engagement with the tapered entry to the inlet of the adaptor.

The point-of-care testing cartridge then is removed from the manufacturer's package. Many manufacturers of testing cartridges provide a hinged cover for the entry port that is rotated into a covering disposition over the entry port. Thus, the cover, if present, must be rotated away from the entry port of the testing cartridge. The outlet end of the adaptor tube then is urged toward the entry port of the testing cartridge in an orientation that permits the guide walls of the adaptor to align with and nest with the side walls of the testing cartridge in proximity to the entry port. The relative length of the guide walls and the adaptor tube ensure that the guide walls engage the side walls of the testing cartridge before the adaptor tube contacts either the top wall or the entry port. Additionally, the guide walls are offset latterly from the adaptor tube distances appropriate for guiding the outlet end of the adaptor tube precisely into the entry port of the testing cartridge. The walls of the adaptor hold the adaptor and the syringe in a stable orientation relative to the testing cartridge. The plunger of the syringe assembly then is moved distally to urge a selected volume of the specimen from the fluid receiving chamber of the syringe body, through the adaptor and into the testing cartridge. The syringe assembly and the adaptor then are removed from the testing cartridge and are discarded in a conventional safe manner. The tip cap, if provided, may be rotated over the outlet section of the adaptor to prevent leakage or spillage as the syringe assembly and adaptor are being transported to a disposal receptacle. Simultaneously, the cover of the testing cartridge is rotated over the entry port of the testing cartridge, and the testing cartridge is presented to a portable clinical analyzer substantially in the conventional manner. As an alternate to the above-described procedure, the testing cartridge may be engaged with the portable clinical analyzer before depositing the specimen in the testing cartridge.

### DESCRIPTION OF THE DRAWINGS

FIG. 1 is a perspective view of an adaptor in accordance with the subject invention.

FIG. 2 is a front elevational view of the adaptor.

FIG. 3 is a bottom plan view of the adaptor.

FIG. 4 is a cross-sectional view taken along line 4-4 in FIG. 2.

FIG. 5 is an exploded perspective view of a syringe for use with the adaptor shown in FIGS. 1-4.

FIG. 6 is a perspective view of a testing cartridge for use with the adaptor.

FIG. 7 is a perspective view of the adaptor mounted to the syringe.

FIG. 8 is an exploded cross-sectional view showing the adaptor and syringe guided toward the inlet port of the testing cartridge.

FIG. 9 is a perspective view showing the adaptor and syringe fully mounted on the testing cartridge for delivering a specimen to the testing cartridge.

FIG. 10 is a cross-sectional view similar to FIG. 8, but showing an alternate embodiment of the adaptor.

FIG. 11 is a perspective view similar to FIG. 9, but showing the alternate adaptor.

FIG. 12 is a perspective view similar to FIG. 7, but showing the alternate adaptor with the tip cap closed.

### DETAILED DESCRIPTION

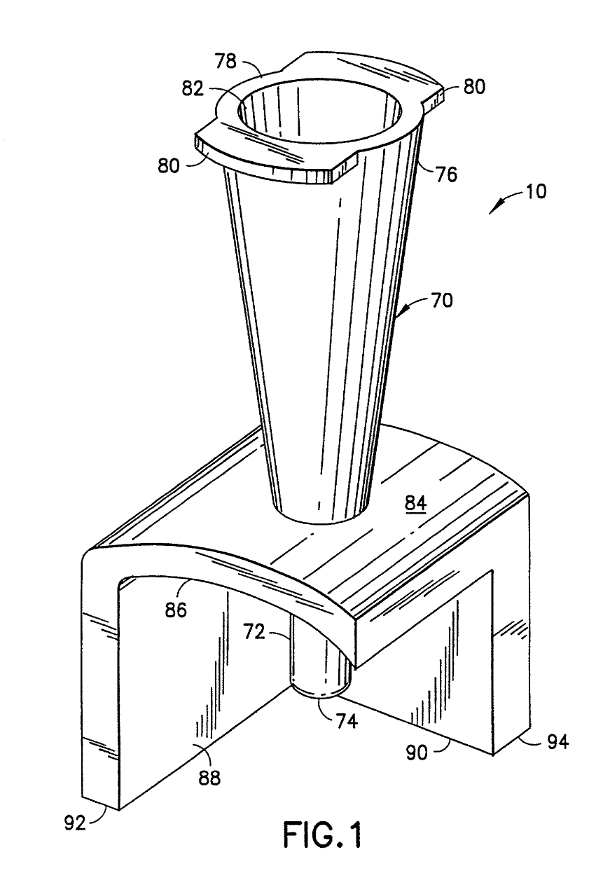

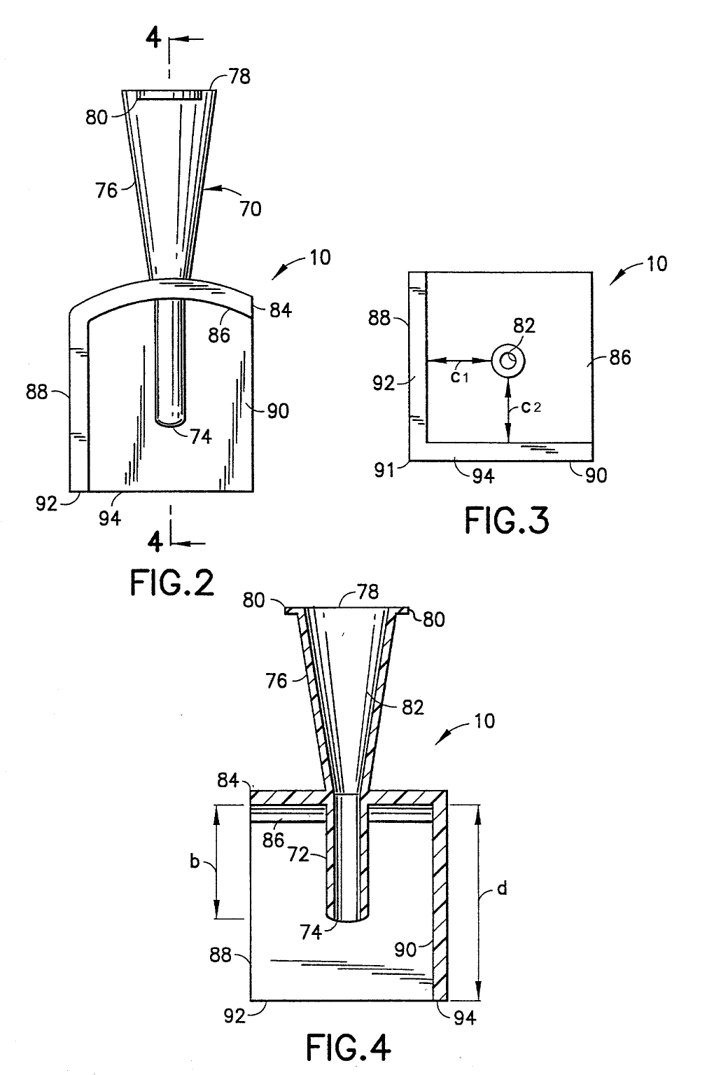

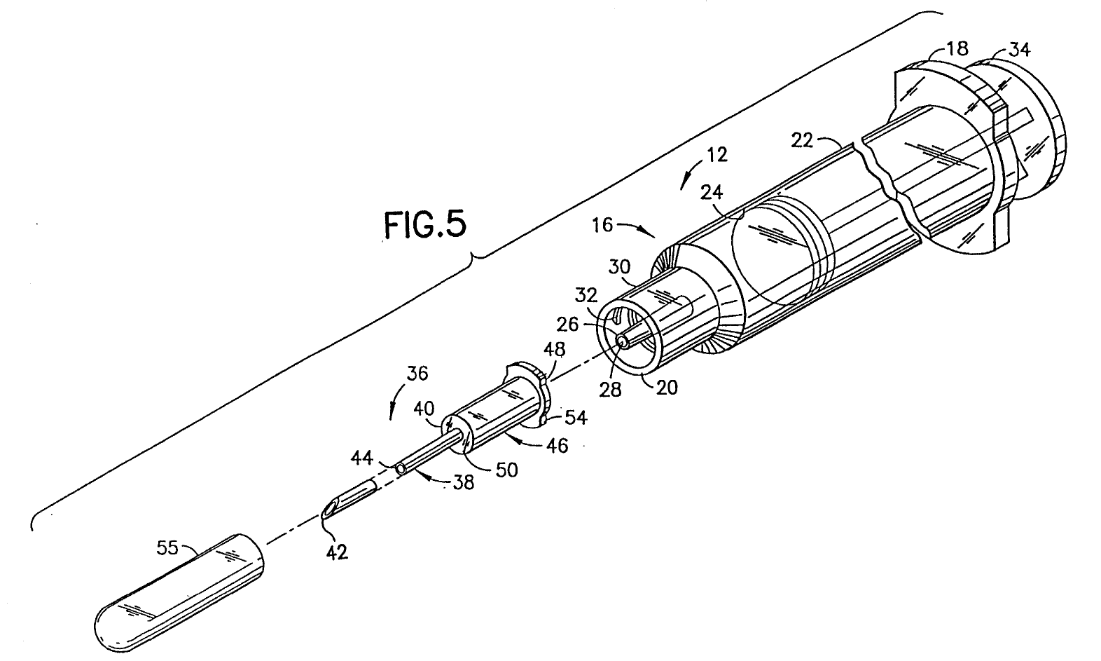

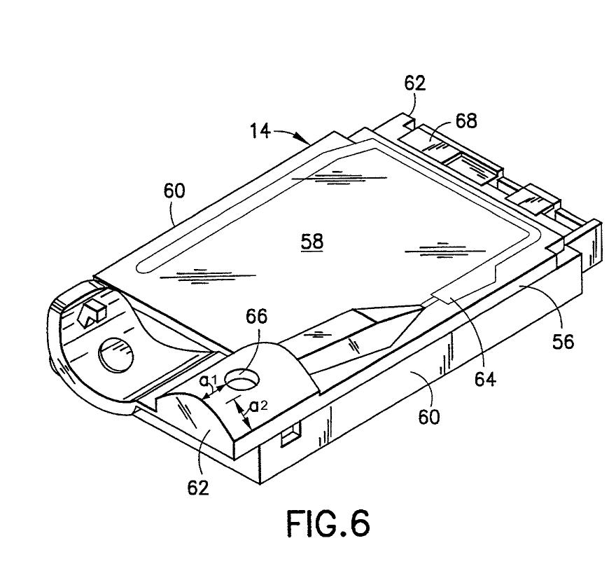

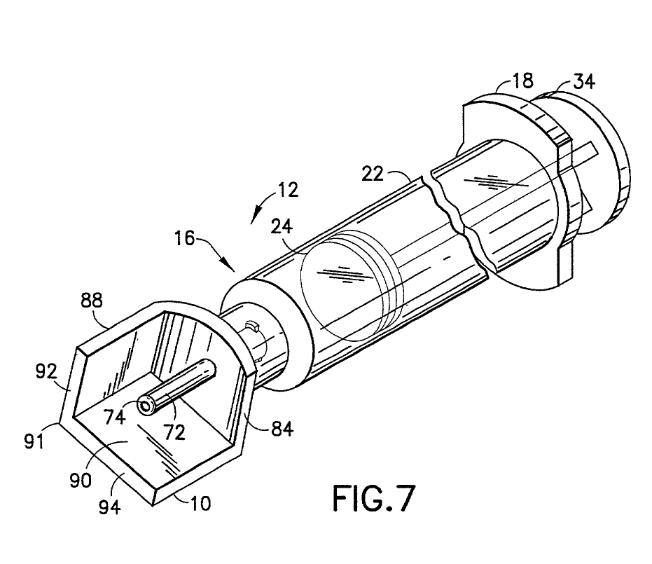

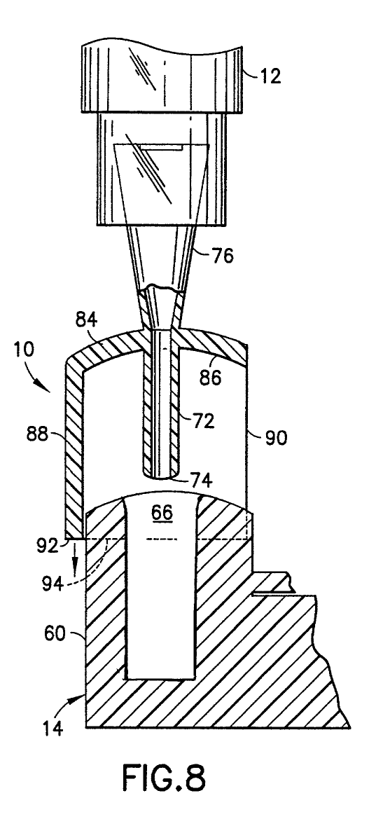

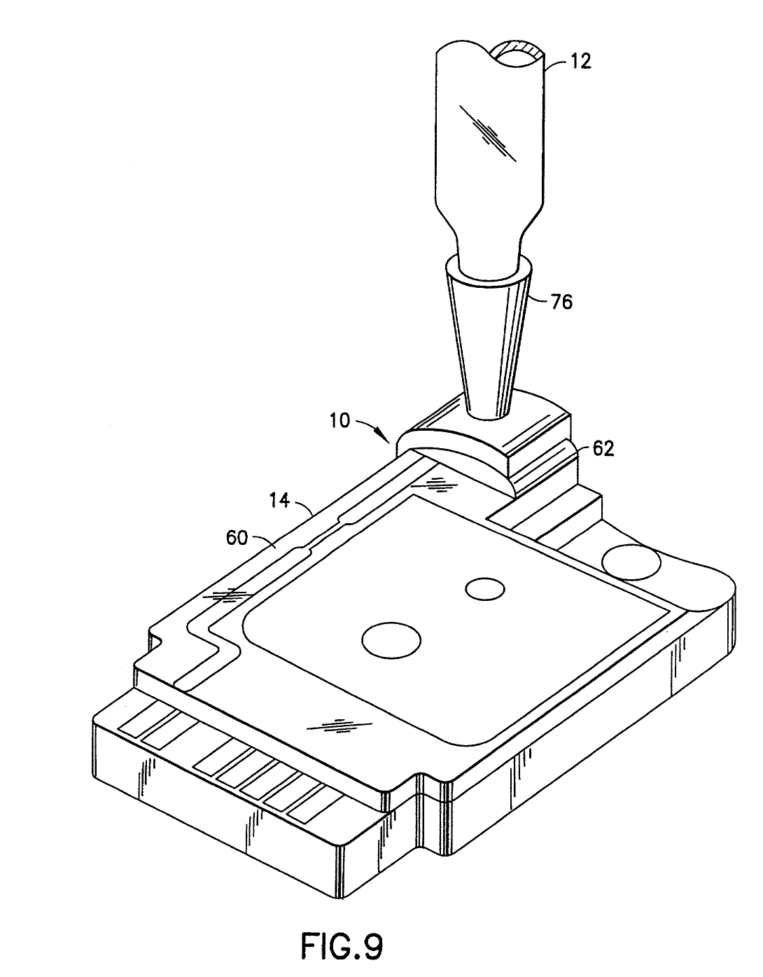

An adaptor in accordance with the subject invention is identified generally by the numeral 10 in FIGS. 1-4. Adaptor 10 is used with a syringe assembly 12, as shown most clearly in FIG. 5, and with a point-of-care testing cartridge 14, as shown most clearly in FIG. 6.

Syringe assembly 12, as shown in FIG. 5, includes a syringe body 16 having a proximal end 18 and a distal end 20. A barrel 22 extends distally from proximal end 18 and defines a cylindrical fluid receiving chamber 24 that is widely open at proximal end 18. A frustoconically tapered Luer tip 26 extends from barrel 22 to distal end 20 of syringe body 16. Tip 26 is provided with a narrow cylindrical passage 28 that communicates with fluid receiving chamber 24 of barrel 22. An optional Luer collar 30 projects distally from barrel 22 and concentrically surrounds Luer tip 26. Luer collar 30 is provided with an internal array of threads 32. Syringe assembly 12 further includes a plunger 34 slideably disposed in fluid receiving chamber 24 and in fluid-tight engagement with the cylindrical walls of chamber 22. Plunger 34 can be moved alternately in proximal or distal directions for urging fluid through passage 28 in tip 26 and into or out of fluid receiving chamber 24.

Syringe assembly 12 optionally includes a needle assembly 36. Needle assembly 36 includes a metallic needle cannula 38 having a proximal end 40, a sharply pointed distal end 42 and a lumen 44 extending between the ends. Needle assembly 36 further includes a hub 46 that has a proximal end 48, a distal end 50 and a passage extending therebetween. Distal end 50 of hub 46 is securely mounted to proximal end 40 of needle cannula 38 such that the passage through hub 46 communicates with lumen 44 through needle cannula 38. The passage of hub 46 defines a taper that substantially matches tapered Luer tip 26 on syringe body 16. Thus, tapered Luer tip 26 of syringe body 16 can be placed in fluid-tight frictional engagement with the passage in proximal end 48 of hub 46. Proximal end 48 of hub 46 is further characterized by a pair of diametrically opposite lugs 54 that are dimensioned and configured for engagement with threads 32 of Luer collar 30. Thus, lumen 44 through needle cannula 38 can be placed in communication with passage 28 in Luer tip 26 and with fluid receiving chamber 24 of syringe body 16. Needle assembly 36 further includes a protective cap 55 removably engaged over needle cannula 38.

Point-of-care testing cartridge 14 is shown in FIG. 6 and may be of any of several prior art designs, including those manufactured by i-STAT Corporation, Diametrics Medical, Inc., AVL Scientific Corporation or any other such testing cartridges that are available or become available. One such testing cartridge is disclosed in U.S. Pat. No. 5,638,828, the disclosure of which is incorporated herein by reference.

Testing cartridge 14 includes a generally rectangular body 56 with a top wall 58 that has a length of approximately 1.5-2.0″ and a width of about 1.0″. Body 56 further has side walls 60 and end walls 62 that define a thickness for body 56 of about 0.25″. A fluid reservoir 64 is formed inside body 56 of cartridge 14 and has a volume in the range of 40 μl and 125 μl. Body 56 further includes an entry port 66 that extends through top wall 58 and communicates with reservoir 64. Entry port 66 is slightly tapered from a relatively large diameter portion externally on housing 56 to a relatively smaller cross-section closer to reservoir 64. Additionally entry port 66 is spaced from one side wall 60 and one end wall 62 by a distances “a1” and “a2” respectively. Testing cartridge 14 further includes contact pads and sensors 68 that can be placed in communication with a portable clinical analyzer for performing various point-of-care diagnostic tests on the sample of blood in the reservoir 64 and for providing various readout data that can be used by a health care technician at the point-of-care and/or at a remote location.

Adaptor 10 is molded unitarily from a transparent plastic material and includes a tapered tube 70 that has a narrow cylindrical outlet section 72 with a slightly rounded or tapered outlet end 74. Outlet section 72 is diametrically smaller than entry port 66 of testing cartridge 14. Thus air in reservoir 64 can be vented easily as liquid is deposited therein. Furthermore, the diametrically small outlet provides precise control of fluid flow through tapered tube 70. Tapered tube 70 further includes a tapered female Luer fitting 76 that is substantially concentric with outlet section 72. Luer fitting 76 includes an inlet end 78 with a pair of diametrically opposite Luer lugs 80 that are dimensioned and configured for threaded engagement with threads 32 on the optional Luer collar 30 of syringe assembly 12. However, not all syringes include a Luer collar, and an adaptor for use with syringes that have no Luer collar need not be provided with lugs 80. Adaptor 10 further includes a passage 82 extending axially from inlet end 78 to outlet end 74. Portions of passage 82 adjacent inlet end 78 are conically tapered for fluid-tight engagement with Luer tip 26 of syringe body 16.

Adaptor 10 further includes a support wall 84 that extends substantially transverse to tapered tube 70 at a location between inlet end 78 and outlet end 74 and spaced from outlet end 74 by a distance “b”. Support wall 84 has a supporting surface 86 that is configured to nest with portions of testing cartridge 14 surrounding entry port 66. In the illustrated embodiment, supporting surface 86 of support wall 84 is a substantially concave cylindrically generated surface. The distance “b” is selected to prevent outlet end 74 from being closed off by the bottom of entry port 66.

Adaptor 10 further includes guide walls 88 and 90 that project down from support wall 84 and substantially parallel to outlet section 72. Guide walls 88 and 90 are substantially perpendicular to one another and are unitarily joined at a corner 91. As shown most clearly in FIG. 3, guide walls 88 and 90 are spaced from outlet section 72 by a distances “c1” and “c2” that substantially equals the distance “a1” and “a2” between entry port 66 of testing cartridge 14 and the adjacent side walls 60 and end walls 62 of body 56 of testing cartridge 14. Guide walls 88 and 90 include bottom surfaces 92 and 94 respectively. Bottom surfaces 92 and 94 define a plane substantially orthogonal to tapered tube 70. Additionally, the plane defined by bottom surfaces 92 and 94 is spaced from support wall 84 by a distance “d” that exceeds the projection “b” of outlet end 74 from support wall 84.

Syringe assembly 12 is used in a conventional manner to draw a sample of fluid from a patient. More particularly, needle assembly 36 can be mounted to Luer tip 26 of syringe body 16, and needle cannula 38 of needle assembly 36 can be inserted into a blood vessel of a patient or other source of bodily fluid for drawing a sample of blood or other such fluid. Alternatively, a blunt plastic cannula or other plastic Luer fitting can be mounted to Luer tip 26, and the distal end of the blunt plastic cannula or other fitting can be urged through the septum that seals a fitting of a fluid collection set. Still further, syringe assembly 12 can be connected directly to an arterial or venous line that had already been placed in communication with a patient. With any of these optional approaches, plunger 34 is moved proximally after accessing the supply of fluid. Proximal movement of plunger 34 draws fluid into fluid receiving chamber 24 of syringe barrel 22. The volume of fluid drawn into fluid receiving chamber 24 is in excess of the volume of fluid required for testing cartridge 14, which typically is in the range of 40 μl-125 μ. Needle assembly 36 or the blunt plastic cannula, if used, then is removed from syringe body 16 substantially in a conventional manner and is disposed of in a sharps receptacle.

Adaptor 10 then is mounted to Luer tip 26. More particularly, Luer tip 26 is axially aligned with inlet end 78 of Luer fitting 76 of adaptor 10. In the illustrated embodiments, syringe assembly 12 includes a Luer collar 30, and adaptor 10 includes lugs 80 that are dimensioned for engagement with threads 32 of Luer collar 30. Thus, in this embodiment adaptor 10 is rotated for threaded engagement of lugs 80 with threads 32 of Luer collar 30. This threaded engagement causes Luer tip 26 of syringe body 16 to be urged into fluid-tight engagement with conically tapered portions of passage 82 adjacent inlet end 78 of adaptor 10. Other syringes, however, may not have a Luer collar. For these embodiments, adaptor 10 need not have lugs 80 or lugs 80 need not be utilized. Thus, the conically tapered tip of a syringe without a Luer collar can merely be urged axially into fluid-tight frictional engagement with conically tapered surfaces of passage 82 adjacent inlet end 78.

Point-of-care testing cartridge 14 then is removed from the manufacturer's package, and any closure that may have been positioned over entry port 66 is rotated away from entry port 66. Syringe assembly 12 and adaptor 10 then are moved toward entry port 66 of testing cartridge 14. More particularly, guide walls 88 and 90 are urged into sliding engagement with side and end walls 60 and 62 of body 56 of testing cartridge 14 in proximity to entry port 66. As noted above, guide walls 88 and 90 project further from support wall 84 then outlet port 66. Accordingly, portions of guide walls 88 and 90 in proximity to bottom edges 92 and 94 thereof will guide outlet end 74 of outlet section 72 into entry port 66 of testing cartridge 14. Supporting surface 86 of support wall 84 will prevent over-insertion of outlet section 72, and hence will ensure a smooth delivery of fluid through adaptor 10 and into testing cartridge 14.

The use of testing cartridge 14 proceeds merely by urging plunger 34 distally in syringe body 16. Movement of plunger 34 causes blood in fluid receiving chamber 24 to be urged through Luer tip 26 of syringe body 16, through passage 82 of adaptor 10 and into reservoir 64 of testing cartridge 14. Syringe assembly 12 and adaptor 10 then are separated from testing cartridge 14. The cover of testing cartridge 14 then is rotated into the closed position and syringe assembly 12 and adaptor 10 are discarded in a safe accepted manner.

The small diameter of outlet section 72 of tapered tube 70 provides very precise fluid flow from syringe assembly 12 and prevents underfilling or overfilling of reservoir 64. Furthermore, the dimensions of outlet section 72 relative to entry port 66 ensures efficient venting, and hence avoids the creation of bubbles in the sample. Additionally, the length of outlet section 72 from supporting surface 86 prevents outlet section 72 from bottoming out in entry port 66 and hence achieves a smooth bubble-free flow. Still further, the transparent plastic of adaptor 10 provides visual feedback that enables more accurate flow control.

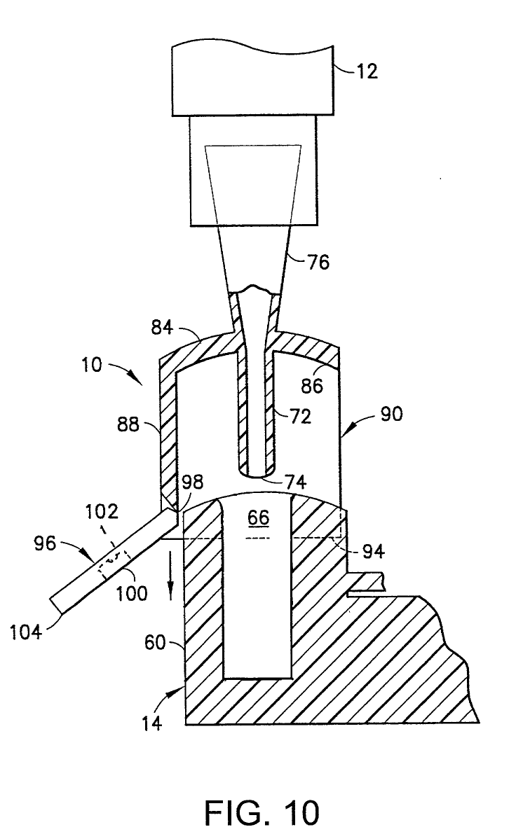

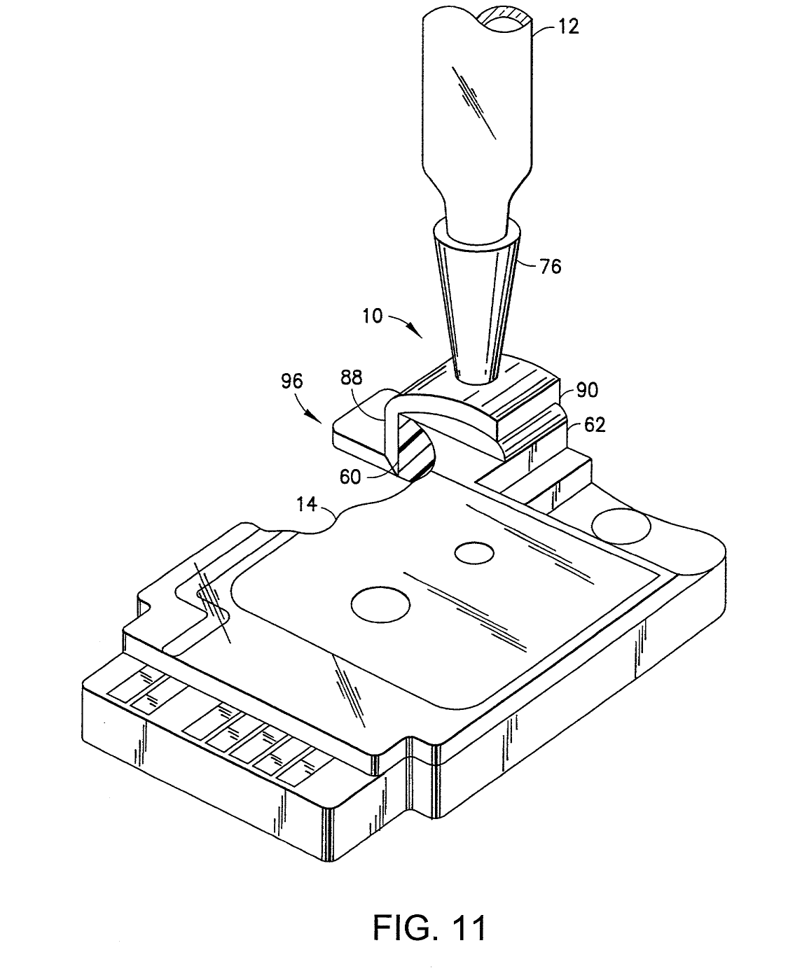

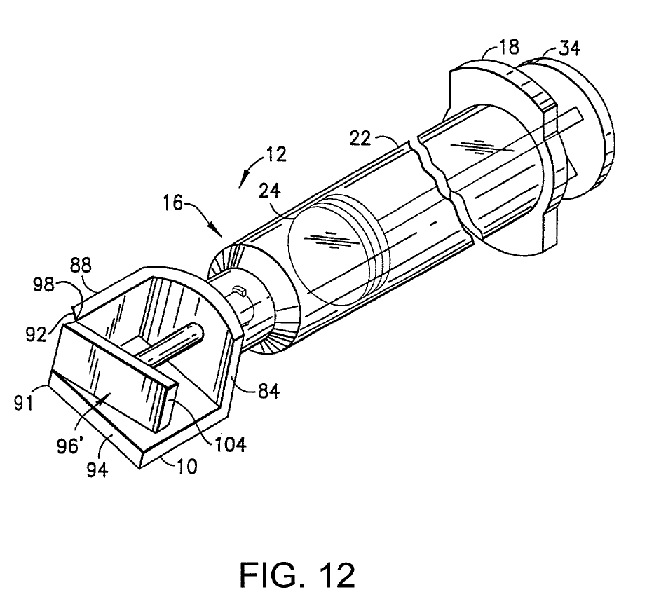

An alternate adaptor in accordance with the invention is illustrated in FIGS. 10-12. The alternate adaptor is virtually identical to the adaptor described above and illustrated in FIGS. 1-4. Accordingly, corresponding elements of the alternate adaptor merely have been identified by the same reference numerals as the adaptor shown in FIGS. 1-4, and a further description of those elements is omitted. The alternate adaptor differs from the first embodiment in that a tip cap 96 is hingedly articulated to guide wall 88 by a living hinge 98. Thus, tip cap 96 is molded unitarily with remaining portions of the adaptor. Living hinge 98 enables tip cap 96 to be rotated between an open condition, as shown in FIGS. 10 and 11 and a closed condition, as shown in FIG. 12. As shown in FIG. 10, tip cap 96 includes a surface 100 that can be rotated into opposed facing relationship without outlet end 74 of tube 70. Surface 100 is formed with a recess 102 dimensioned for snapped frictional engagement over outlet section 72 of tapered tube 70 for sealing passage 82 through tapered tube 70. However, a digital force exerted on an edge 104 of tip cap 96 opposite living hinge 98 can release tip cap from outlet section 72 of tapered tube 70 for rotating tip cap from the closed condition shown in FIG. 12 to the open condition shown in FIGS. 10 and 11.

The alternate adaptor is used substantially in the same manner as the adaptor described and illustrated above. The tip cap 96 can be rotated into an unobtrusive position, as shown in FIGS. 10 and 11 when fluid is being transferred to the testing cartridge. After use, or between uses, tip cap 96 is rotated into the position shown in FIG. 12.

What is claimed is:

1. An adaptor for use with a testing cartridge and a syringe to facilitate delivery of a fluid specimen from said syringe to said testing cartridge, said adaptor comprising a tapered tube having an outlet end, an inlet end and a passage extending between said ends, said outlet end being cross-sectionally smaller than said inlet end, a support wall extending from said tube at a location between said ends such that said outlet end of said tube projects in an outlet direction beyond said support wall, and at least one guide wall projecting in said outlet direction from said support wall a distance greater than the projection of said outlet end of said tube from said support wall.

2. The adaptor of claim 1, wherein said at least one guide wall comprises two angularly aligned guide walls.

3. The adaptor of claim 2, wherein said testing cartridge includes and entry port, said outlet of said tube being cross-sectionally smaller than said entry port of said testing cartridge to achieve efficient venting of air from said testing cartridge.

4. The adaptor of claim 3, wherein said testing cartridge comprises a housing having a top wall and at least a pair of intersecting side walls extending from said top wall, said entry port of said testing cartridge extending through said top wall at a location in proximity to said intersecting side walls, said guide walls of said adaptor being aligned for slidable engagement with the said side walls of the testing cartridge, and said outlet end of said tube being spaced from said guide walls sufficiently for alignment with said entry port of said testing cartridge when said guide walls are engaged with said side walls of said testing cartridge.

5. The adaptor of claim 4, wherein portions of said the top wall of said testing cartridge in proximity to said entry port are convexly arcuate, portions of said support wall surrounding said outlet of said tube being concavely arcuate and configured for nesting with said convexly arcuate portions of said top wall of said testing cartridge surrounding said entry port to said testing cartridge.

6. The adaptor of claim 4, wherein said tube, said support wall and said guide walls are unitarily molded from a plastic material.

7. The adaptor of claim 1, wherein said syringe includes a Luer tip, and wherein said inlet of said tube of said adaptor is tapered for fluid-tight mating with said Luer tip of said syringe.

8. The adaptor of claim 7, wherein said syringe includes an internally threaded Luer collar surrounding said Luer tip, said inlet end of said tube of said adaptor comprising a pair of opposite Luer projections for threaded engagement with said Luer collar.

9. The adaptor of claim 1, further comprising a tip cap hingedly connected to portions of said guide wall opposite said support wall, said tip cap being hingedly rotateable between a position spaced from said outlet end and a position in sealing engagement with said outlet end.

10. The adaptor of claim 9, wherein said tip cap is unitarily connected to said guide wall by a living hinge.

11. The adaptor of claim 9, wherein said tip cap includes an aperture extending into a surface thereof, said aperture being dimensioned and disposed for sealing frictional engagement around said outlet end of said tube when said tip cap is in said closed position.

12. The adaptor of claim 9, wherein said tube, said support wall, said guide walls and said tip cap are unitarily molded from a plastic material.
